(19) United States
(12) Patent Application Publication
(10) Pub. No.: US 2026/0176678 A1
(43) Pub. Date: Jun. 25, 2026

(54) RECOVERING SELECTED PARTITIONS IN A DIGITAL ASSAY

(71) Applicant: MelioLabs Inc., Santa Clara, CA (US)

(72) Inventors: Luc Bousse, Santa Clara, CA (US); Mridu Sinha, Santa Clara, CA (US); Rachit Shrivastava, Santa Clara, CA (US); Joshua Kannankunni, Santa Clara, CA (US); Evan Baerg, Santa Clara, CA (US)

(21) Appl. No.: 19/541,213

(22) Filed: Feb. 16, 2026

Related U.S. Application Data
(63) Continuation of application No. PCT/US2024/042125, filed on Aug. 13, 2024.
(60) Provisional application No. 63/520,175, filed on Aug. 17, 2023.

(51) Int. Cl. C12Q 1/6851

(52) U.S. Cl. CPC C12Q 1/6851

(57) ABSTRACT

Provided are devices, systems, and methods for retention of partition during biological analysis of a sample with digital assay. The method for biological analysis of a sample includes, for example, a substrate with an array with at least 10,000 individual partitions; division of the sample by loading aliquot of the sample into the individual partitions; perform a biological analysis on each partition to generate a result for each partition; use the results of the individual partitions to select one or more of the individual partitions to be retrieved; and retrieving the contents of the selected partitions by the application of a focused vacuum or pressure device by measuring and comparing the retrieval pressure on the partition.

### CROSS-REFERENCES TO RELATED APPLICATIONS

The present application is a continuation application of PCT/US2024/042125, filed on Aug. 13, 2024, which claims priority to U.S. Provisional Pat. Appl. No. 63/520,175, filed Aug. 17, 2023, the disclosures of which are incorporated herein by reference in their entirety.

### FIELD

The present disclosure is related to devices, systems, and methods for the recovery of selected partitions in a digital assay. More particularly, the devices, systems, and methods include the biological analysis of a sample in the partitions to identify and separate the contents of the partition by applying retrieval pressure onto the partitions using a focused vacuum or pressure device.

### BACKGROUND

Biological analysis methods, especially genetic or nucleic acid analysis methods, in a digital format have been found to have multiple advantages. In a digital format, a sample is divided in a large number of partitions, in each of which an analysis or an assay is performed. The main advantages of digital assays are the possibility of absolute quantification, without the need for standard curves, and the reduction of background interferences due to the partitioning.

### SUMMARY

Embodiments provided herein relate to devices, systems, and methods for recovering of partitions containing biological samples using digital assay.

Some embodiments provided herein relate to methods for biological analysis of a sample. In some embodiments, the methods include providing a substrate that includes an array having individual partitions, each partition including a hole suitable to allow filling of each partition by capillary force. In some embodiments, the methods include dividing the sample by loading an aliquot of the sample into the individual partitions. In some embodiments, the methods include performing a biological analysis on each partition. In some embodiments, the methods include identifying one or more individual partitions of interest based on the biological analysis. In some embodiments, the methods include retrieving the contents of the one or more partitions of interest. In some embodiments, the array includes at least 10,000 individual partitions.

In some embodiments, retrieving the contents of the one or more partitions of interest includes application of a focused vacuum or pressure device. In some embodiments, the focused vacuum or pressure device exerts a retrieval pressure on the partition greater than a capillary retention pressure of the partition. In some embodiments, the retrieval pressure is calculated based on a dimension of the partition. In some embodiments, the retrieval pressure is equal to or greater than a minimal retrieval pressure. In some embodiments, the retrieval pressure is at least 50% higher than the minimal retrieval pressure. In some embodiments, the retrieval pressure is at least 100% higher than the minimal retrieval pressure. In some embodiments, the vacuum or pressure device includes a probe having dimensions smaller than the dimensions of the partition. In some embodiments, the focused vacuum or pressure device includes a probe or a needle for applying the retrieval pressure.

In some embodiments, the individual partitions are circular. In some embodiments, the individual partitions are hexagonal.

In some embodiments, the methods further include applying a protective layer. In some embodiments, the protective layer includes a water-immiscible liquid. In some embodiments, the protective layer includes a fluorinated oil. In some embodiments, the protective layer further includes one or more polymer layers.

In some embodiments, the individual partitions have an area density of at least 30%. In some embodiments, the biological analysis includes nucleic acid amplification. In some embodiments, the biological analysis includes a melt curve analysis. In some embodiments, the sample includes whole cells or microorganisms.

In some embodiments, a location of the individual partition of interest is determined by a computer vision system. In some embodiments, the computer vision system utilizes one or more visual references in the array.

### BRIEF DESCRIPTION OF THE DRAWINGS

In order to describe the manner in which the above-recited and other advantages and features of the present disclosure can be obtained, a more particular description will be rendered by reference to specific embodiments thereof which are illustrated in the appended drawings. Understanding that these drawings depict only typical embodiments and are not therefore to be considered to be limiting of its scope, the disclosure will be described and explained with additional specificity and detail through the use of the accompanying drawings in which:

FIG. 1 illustrates an embodiment of a silicon chip etched with dense array of high aspect ratio holes.

FIG. 2 illustrates an embodiment of a microscope image, at high magnification, of a portion of an etched silicon chip with a reference pattern.

FIG. 3 illustrates an embodiment of a layout of an area of the photomask design with three design dimensions.

FIG. 4 illustrates an embodiment of a cross-section of a group of holes which are 52 μm wide and are separated by 20 μm walls.

FIG. 5 illustrates an embodiment of an aqueous sample in the process of being removed in the upwards direction and the surface tension force between the aqueous sample, the second immiscible fluid, and the wall of the partition.

FIG. 6 illustrates an embodiment of an experimental set up describing a method of biological analysis.

FIG. 7 illustrates an embodiment of a retention of a cluster of wells when a blunt and/or large tip is used on the partitions.

FIG. 8 illustrates an embodiment of a retention of a small cluster of wells when using a fine tip on the partitions.

FIG. 9 illustrates an embodiment of an experimental set up describing the claimed method of biological analysis using a miniature vacuum pump that can be controlled using a vacuum regulator or by adjusting voltage applied to the vacuum pump.

FIG. 10 illustrates an embodiment of a complete retention of a single well and partial retention of two neighboring wells.

FIG. 11A illustrates an embodiment of an array filled with fluorescent ROX dye, prior to retention of a partition.

FIG. 11B illustrates an embodiment of an array filled with fluorescent ROX dye, following complete retention of a well.

FIG. 11C illustrates an embodiment of a video frame at the moment that the fluorescent dye moves into the pickup needle.

FIG. 12 illustrates an embodiment of a result of an experiment without the use of an oil protective layer. The sample pick up is less effective.

FIG. 13 illustrates an embodiment of an experimental sequence of single well retention with a reference pattern on the silicon chip having a group of locations in the array without any etching.

FIG. 14 illustrates an embodiment of a silicon chip loaded with a sample containing C. parapsilosis after thermal cycling with bright wells indicating amplification.

FIG. 15A illustrates an embodiment of a region of the silicon chip with a cluster of wells selected for further analysis.

FIG. 15B illustrates an embodiment of the control melt curves and melt curves of the circled wells of FIG. 15A.

FIG. 16A illustrates an embodiment of the melt curves after qPCR of the samples retrieved from eight wells of FIG. 15A.

FIG. 16B illustrates an embodiment of the melt curves of qPCR of the control samples containing cyanobacteria DNA.

### DETAILED DESCRIPTION

Unless otherwise defined, all technical and scientific terms used herein have the same meaning as commonly understood by one of ordinary skill in the art. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety herein. In case of conflict, the present specification, including definitions, will control. Other features and advantages will be apparent from the following detailed description and figures, and from the claims.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As used herein, the term “biological sample” or “sample” means a material, substance, or solution comprising one or more biological molecules, chemicals, components, and/or compounds (e.g., a nucleic acid, DNA molecule, or RNA molecule) of interest to a user, manufacturer, or distributor of the various embodiments described or implied herein. A sample may include, but is not limited to, one or more of a DNA sequence (including cell-free DNA), an RNA sequence, a gene, an oligonucleotide, an amino acid sequence, a protein, a biomarker, or a cell (e.g., circulating tumor cell), or any other suitable target biomolecule. As used herein, the term “sample solution” means a liquid or fluid comprising at least one sample.

As used herein, the term “biological analysis” means the analysis of a biological substance in order to ascertain its influence on living organisms. It is scientific approach that combines analytical tools and biological content in one place to enhance deeper and broader understanding of biological relationships and processes to experimental observations and translation of that understanding to hypothesis. Biological analysis is typically done on mRNA, miRNA, protein, SNP, metabolites or any other biomolecule by performing basic statistics such as background correction, normalization, p-value, and so on.

As used herein, the term “capillary force” means a force required by a liquid flowing in a narrow space without the assistance of, or even in opposition to, any external forces like gravity. Capillary force acts at fluid-air-solid interfaces to minimize the surface energy of the interface. It is vital for moving the liquid phases in the upward direction; the movement of liquid phase within the porous space is regulated by forces of adhesion, cohesion, and surface tension.

As used herein, the term “capillary pressure” means the pressure between two immiscible fluids in a thin tube, resulting from the interactions of forces between the fluids and solid walls of the tube. For example, if a small tube in water is overlaid by oil, water will rise up into the tube due to capillary pressure.

As used herein, the term “selected” in reference to selected partitions of biological sample during analyses has its ordinary meaning as understood in light of the specification, and refers to the intentional discrimination of a particle of interest. Thus, a selectively enriched sample is a sample that includes a particle or target of interest, but that does not include, or does not substantially include, other particles.

The present disclosure is based, in part, on the development of methods for retrieval of one or more samples from a fixed partition of a multipartition format.

In some embodiments, a fixed partition with a fixed location in space is known beforehand. The use of fixed partitions has several advantages. First, each partition is separated in space and does not touch the other partitions, unlike droplets which are free to touch each other. This reduces the risk of exchange or interactions between the partitions. Second, detection is improved because the location of each sample is known, and as much time as needed can be used for detection. Third, the use of fixed partitions in known locations enables the possibility of selectively recovering certain partitions for further analysis. One method of dividing a sample into fixed partitions uses open holes, or wells open on two sides that are filled by capillary forces, or surface tension forces. However, to benefit from the advantages of fixed partitions, it may be necessary to selectively remove one or more products from an identified fixed partition. There remains a need to develop methods that are efficient in recovery of such products, particularly from fixed partitions that use open holes, or wells open on two sides.

Multiple methods of creating fixed partitions exist. One type of method uses chambers or wells connected by one or more flow channels that are filled with the sample, followed by flowing in an immiscible fluid to perform the separation into distinct partitions. Another method uses open holes, or wells open on two sides that are filled by capillary forces, or surface tension forces. In all cases, after creating the partitions, it is useful to isolate them from each other, and protect them from evaporation and the outside environment by using one or more protective layers. These layers could be composed of an immiscible liquid, such as an oil, mineral oil, silicone oil, fluorinated liquids such as FC-40 or HFE 7500, or water-immiscible esters. Other possible protective layers that could be applied are solid, such as polymers, including thermoplastic polymer films, polydimethylsiloxane, other types of rubber films or septa, metal coated polymer films, and so forth.

After a sample is partitioned, each partition is subjected to a biological assay. One common type of assay is nucleic acid amplification such as polymerase chain reaction (PCR), and in that case the method is known as digital PCR (dPCR). Many other types of assay are also possible. One example is the analysis of intact cells or microorganisms, where the partitions contain whole cells. More details of this type of analysis can be found in U.S. Pat. No. 11,174,504.

In some embodiments, the biological assay can be an isothermal amplification or loop mediated isothermal amplification (LAMP), such as in which the DNA amplification takes place at constant temperature as opposed to a series of alternating temperatures in PCR reaction.

As used herein, the term “amplification product” means any product produced by an amplification assay or process, for example, an increased number of target nucleic acid molecules or other nucleic acid molecules produced during a PCR assay or process (e.g., during a qPCR or dPCR assay or process). As used herein, an “indicator of amplification” means a physical, electrical, magnetic, chemical, and/or optical property or effect produced by a sample that may be used in determining the existence, and/or in determining, measuring, or estimating an amount of amplification of a target nucleic acid in a biological assay, test, process, or experiment (e.g., a PCR assay, test, process, or experiment). An indicator of amplification may comprise one or more of luminescence (e.g., fluorescence, chemiluminescence, bioluminescence), color, transmissivity, opacity, reflectivity, polarization, pH, charge, surface potential, current, or voltage changes.

Polymerase chain reaction (PCR) may comprise a thermal cycling process, in which cycles of heating and cooling are used to provide repeated cycles of nucleic acid melting and enzymatic replication of nucleic acids. A number of PCR methods use thermal cycling involving alternately heating and cooling the PCR sample to a defined series of temperature steps. These thermal cycling steps may be used first to physically separate nucleic acids, such as separating the two strands in a nucleic acid double helix, at a high temperature in a process called melting. At a lower temperature, each strand is then used as the template in synthesis by the polymerase to selectively amplify a target nucleic acid during an annealing phase and extension phases. Example polymerases include heat-stable polymerase such as, for example, Taq polymerase. The selectivity of PCR results from the use of primers that are complementary to nucleic acid regions targeted for amplification under specific thermal cycling conditions. Primers (short nucleic acid fragments) containing sequences complementary to the target region along with a polymerase, are used to enable selective and repeated amplification.

Digital PCR (dPCR) is a refinement of conventional polymerase chain reaction (PCR) methods which can be used to directly quantify and clonally amplify nucleic acids (including DNA, cDNA, methylated DNA, RNA, or the like). One difference between dPCR and traditional PCR lays in the method of measuring nucleic acids amounts. In dPCR, a sample is separated into a large number of individual sample volumes or portions, and respective PCR reactions are carried out in each sample portion individually. This separation allows for sensitive measurement of small amounts of a nucleic acid. dPCR has been demonstrated as useful for studying variations in gene sequences, such as copy number variation or point mutations.

In dPCR, a sample is partitioned so that individual nucleic acid molecules to be assessed within the sample are localized and concentrated within many separate regions. While the starting number of copies of a molecule is proportional to the number of amplification cycles in conventional PCR, dPCR does not depend on determining a number of amplification cycles to determine the initial sample amount. Instead, the initial sample is partitioned into a large number of relatively small sample portions containing one copy, or approximately one copy, or no copy of the nucleic acid template or target. As a result, each partitioned sample portion may be characterized as a “0” or “1” for containing at least one of a type of target nucleic acid molecule, resulting in a negative (“0”) or positive (“1”) PCR reaction, respectively. The partitioning of the sample in this way may use Poisson statistics to provide an estimate of molecules in the initial sample. However, the accuracy of this estimate varies, depending on the number of “0” and “1” produced.

Systems and methods for performing dPCR are described in the art, including, for example, in US 2017/0088879 and US 2016/0310949, each of which is incorporated by reference herein in its entirety. These references describe the use of dPCR on nucleic acids, such as DNA. However, the methods and systems described therein may be used in the context of the present application for the use of cells and single-cell analysis. For example, in some embodiments, a digital amplification technique is performed. For example, the digital amplification technique may comprise a dPCR assay, process, experiment, or test, wherein a sample or reaction solution is segregated, distributed, or divided, into a plurality of sample reaction volumes or reaction sites associated with a reaction device, fluidic device, sample holder, or other such device. The plurality of sample reaction volumes may include a first plurality of the sample reaction volumes each containing a cell and a second plurality of the sample reaction volumes each containing no cells. The plurality of sample reaction volumes or reaction sites are subjected to an amplification assay using, for example, at least a primer and probe or indicator dye, wherein the amplification assay is configured to amplify a target nucleic acid from the cell. During the dPCR assay, an indicator of the target present in any of the plurality of sample reaction volumes may be detected or measured. Similar to other types of PCR, dPCR may progress by exposing the partitioned sample reaction volumes, which contain reagents for amplification, to an amplification assay designed to amplify the target nucleic acid. For example, thermal cycling may be performed such that the template nucleic acid is amplified within the reaction volumes that include an initial one, or approximately one, copy of the template nucleic acid molecule.

In order to quantify the nucleic acid amplification, an indicator of amplification exhibited by the reaction volumes may be detected. In some exemplary embodiments in accordance with the present disclosure, one or more fluorescent dyes or probes may be used such that the dyes or probes bond to nucleic acids and exhibit fluorescence to indicate presence of a nucleic acid.

For example, amplified target nucleic acids can be detected using a detectable nucleic acid binding agent which can be, for example, an intercalating agent or a non-intercalating agent. As used herein, an intercalating agent is an agent or moiety capable of non-covalent insertion between stacked base pairs of a double-stranded nucleic acid molecule. A non-intercalating agent is one that does not insert into the double-stranded nucleic acid molecule. The nucleic acid binding agent can produce a detectable signal directly or indirectly. The signal can be detectable directly using, for example, fluorescence or absorbance, or indirectly using, for example, any moiety or ligand that is detectably affected by its proximity to double-stranded nucleic acid is suitable, for example a substituted label moiety or binding ligand attached to the nucleic acid binding agent. It is typical for the nucleic acid binding agent to produce a detectable signal when bound to a double-stranded nucleic acid that is distinguishable from the signal produced when that same agent is in solution or bound to a single-stranded nucleic acid. For example, intercalating agents such as ethidium bromide fluoresce more intensely when intercal ated into double-stranded DNA than when bound to single-stranded DNA, RNA, or in solution (see, e.g., U.S. Pat. Nos. 5,994,056; 6,171,785; and 6,814,934). Similarly, actinomycin D fluoresces red when bound to single-stranded nucleic acids, and green when bound to double-stranded nucleic acids. And in another example, the photoreactive psoralen 4-aminomethyle-4-5′,8-trimethylpsoralen (AMT) has been reported to exhibit decreased absorption at long wavelengths and fluorescence upon intercalation into double-stranded DNA (Johnston et al. Photochem. Photobiol. 33:785-791 (1981). For example, U.S. Pat. No. 4,257,774 describes the direct binding of fluorescent intercalators to DNA (e.g., ethidium salts, daunomycin, mepacrine and acridine orange, 4′,6-diamidino-α-phenylindole). Non-intercalating agents (e.g., minor groove binders such as Hoechst 33258, distamycin, netropsin) may also be suitable for use. For example, Hoechst 33258 (Searle, et al. Nucleic Acids Res. 18:3753-3762 (1990)) exhibits altered fluorescence with an increasing amount of target. Exemplary detectable DNA binding agents may include, for example, acridine derivatives (e.g., acridine homodimer, acridine orange, acridine yellow, 9-amino-6-chloro-2-methoxyacridine (ACMA), proflavin), actinomycins (e.g., actinomycin D (Jain, et al. J. Mol. Biol. 68:1-10 (1972), 7-amino-actinomycin D (7-AAD)), anthramycin, auramine, azure B, BOBO™-1, BOBO™-3, BO-PRO™-1, BO-PRO™-3, chromomycin (e.g., A3), crystal violet, cyanine dyes, DAPI (Kapúsciński, et al. Nucleic Acids Res. 6:3519-3534 (1979)), 4′,6-diamidino-2-phenylindole (DAPI), daunomycin, distamycin (e.g., distamycin D), dyes described in U.S. Pat. No. 7,387,887, ellipticine, ethidium salts (e.g., ethidium bromide, ethidium homdimer-1, ethidium homdimer-2, dihydroethidium (also known as hydroethidine), ethidium monoazide), fluorcoumanin, fluorescent intercalators as described in U.S. Pat. No. 4,257,774, GelStar® (Cambrex Bio Science Rockland Inc., Rockland, Me.), hexidium iodide, Hoechst 33258 (Searle, et al., (supra)), Hoechst 33342, Hoechst 34580, homidium, hydroxystilbamidine, JO-JO-1, JO-PRO™-1, LDS 751, LOLO-1, LO-PRO™-1, malachite green, mepacrine (e.g., orange), mithramycin, netropsin, the Nissl substance, 4′,6-diamidino-α-phenylindole, proflavine, POPO™-1, POPO™-3 PO-PRO™-1, propidium iodide, ruthenium polypyridyls, Sevron dyes (e.g., Brilliant Red 2B, Brilliant Red 4G, Brilliant Red B, Orange, Yellow L), SYBR 101, SYBR 102, SYBER 103, SYBR® Gold, SYBR® Green I (U.S. Pat. Nos. 5,436,134 and 5,658,751), SYBR® Green II, SYTOX® Blue, SYTOX® Green, SYTOX® Orange, SYTOR 1, SYTOR 11, SYTOR 13, SYTOR 14, SYTOR 15, SYTOR 16, SYTOR 17, SYTOR 18, SYTOR 20, SYTO® 21, SYTOR 22, SYTOR 23, SYTOR 24, SYTOR 25, SYTOR 40, SYTOR 43, SYTOR 44, SYTOR 45, SYTOR 59, SYTOR 60, SYTOR 61, SYTOR 62, SYTOR 63, SYTOR 64, SYTOR 80, SYTOR 81, SYTOR 82, SYTOR 83, SYTOR 84, SYTOR 85, thiazole orange (Aldrich Chemical Co., Milwaukee, Wis.), TO-PRO-1, TO-PRO-3, TO-PRO-5, TOTO-1, TOTO-2, TOTO™-3, YO-PRO®-1, YO-PRO®-3, YOYO-1, and YOYO®-3 (Molecular Probes, Inc., Eugene, Oreg.), among others.

SYBR® Green I (see, e.g., U.S. Pat. Nos. 5,436,134; 5,658,751; and/or 6,569,927), for example, has been used to monitor an amplification (e.g., PCR) reaction by amplifying the target sequence in the presence of the dye, exciting the biological sample with light at a wavelength absorbed by the dye and detecting the emission therefrom. It is to be understood that the use of the SYBR® Green dye is presented as an example and that many such dyes may be used in the methods described herein. Other nucleic acid binding agents can also be suitable as would be understood by one of skill in the art.

In certain embodiments, detection or measurement of the indicator of amplification in a digital amplification assay may be performed at the endpoint of the amplification reaction. The digital amplification may be detected or measured at an ambient temperature after one or more thermal cycles have been completed. It may be beneficial to detect indicators of amplification at other times (such as during amplification and/or during a melt stage) in order to better determine the amplicons produced.

In some embodiments, the present disclosure contemplates performing a melt stage on the reaction sites after the amplification assay and an end point reading has been performed. During such a melt stage, the sample reaction volumes in the reaction sites are heated at a constant rate over a predetermined time and changes to an indicator of amplification are detected. For example, the plurality of sample reaction volumes may be heated at a constant rate over a period of time, such as 10 minutes, 15 minutes, 30 minutes, 1 hour, or any other suitable period of time. During the heating, changes in an indicator of amplification for the sample reaction volumes may be detected, and changes in the indicators may be identified. For example, the bonds of a nucleic acid molecule may melt causing disassociation during heating. This disassociation may trigger a change (decrease) in the indicator of amplification exhibited.

In some embodiments, a plurality of sample reaction volumes are subjected to an amplification assay. For example, the plurality of sample reaction volumes may be simultaneously subjected to an amplification assay, wherein the amplification assay is designed to amplify the target nucleic acid to produce amplified product (for example, one or more amplicons). The assay may utilize at least a primer, probe and/or dye, and an enzyme, such as a Taqman™ assay or any other suitable assay, as those having ordinary skill in the art are familiar with. Accordingly, the sample reaction volumes contain the sample portion and the reagents for amplification and detection.

In some embodiments, the methods, systems, and assays described herein include two probes, such as a FAM™ dye-labeled probe and a VIC® dye-labeled probe, and amplification detection measurements based on each dye may be utilized in order to determine quantities for amplified target nucleic acid(s). For instance, multiple indicators of amplification may be exhibited from a sample reaction volume based on each of the dye-labeled probes. An assay may also include a variety of primers, such as ELITE® primers. In an embodiment, one ELITE® primer may overlap a target sequence (for example, an allele specific primer) while one ELITE® primer may not (for example, a locus specific primer). Some implementations may leverage a standard primer rather than an ELITe® primer for the locus specific primer. In some embodiments, a multiplexing assay may be used where multiple allele specific primers may generate amplicons with a single locus specific primer.

In some embodiments, an assay may include primers with target specific 3′ domains and non-target specific 5′ tails to generate amplicons with adjusted target melt temperatures. In another example, an assay may include primers with target specific 3′ domains and universal 5′ tails to generate amplicons with adjusted target melt temperatures. In this example, the assay formulations may utilize universal primers such that initial amplification is caused by target specific domains (e.g., target specific 3′ domain) while further amplification can be caused by the universal primers. These amplicons may be later differentiated by target melt temperatures. In some embodiments, an assay may include primers designed to identify amplification reactions involving normal (wild-type) nucleic acids and non-normal (mutant) nucleic acids. An assay may also include primers designed to identify certain types of mutations (for example, single nucleotide polymorphisms (SNPs) and inDels at locus within amplicons). For instance, the identification may be based on target melt temperatures for the produced amplicons. In some embodiments, use of known spike-in concentrations may also be leveraged for identification. Various embodiments may utilize ELITE® primers, non-ELITe® (standard) primers, or any suitable combination.

In some embodiments, the plurality of sample reaction volumes subjected to the amplification assay may be subjected to a plurality of PCR steps, such as thermal cycling, as described herein. For example, temperature of the sample reaction volumes may be increased to physically separate strands of the target nucleic acid (for example, strands of a nucleic acid molecule). The temperature may then be decreased and each strand may be used as a template for synthesis by an enzyme (for example, polymerase) to selectively amplify the target nucleic acid, for instance during annealing and extension phases of the PCR process. In an embodiment, a plurality of PCR cycles may be performed that result in amplification of the target nucleic acid molecule.

In many cases, it is desirable to perform further analysis on certain partitions based on the results of the first assay. For instance, it may be useful to perform Next-Generation Sequencing (NGS), or other sequencing methods such as Sanger Sequencing or shotgun sequencing, on the nucleic acid amplified and characterized in the first assay step in selected partitions in order to obtain the complete sequence of the amplicon. This could be used, for instance, to determine the species of microorganism that corresponds to a particular DNA melting curve detected in the initial digital PCR followed by melt curve analysis (see U.S. Pat. No. 11,174,504), including digital high resolution melt (dHRM). dHRM has its ordinary meaning as understood by those of skill in the art in light of the specification, and refers to digital technique for determining a sequence variation in a double stranded or single stranded nucleic acid by analyzing a melting curve of the nucleic acid as further described in PCT Publication No. WO2018/119443A1, the teachings of which are incorporated herein by reference in their entirety.

Some other examples of biological assays that can be performed downstream after obtaining amplicons from a digital PCR reaction include but are not limited to Library preparation for sequencing, Restriction Fragment Length Polymorphism (RFLP) Analysis to detect genetic mutations such as Single Nucleotide Polymorphism (SLP), mutation detection, molecular cloning, DNA methylation analysis, genotyping, in situ hybridization etc.

In some cases, criteria for the selection of partitions to perform further analysis can be based on the end-point amplification threshold to classify the “rain” associated with digital amplification, where the partition b/w an upper and lower threshold can be sent for further analysis.

In some cases, other criteria for further analysis can be based on melt-curve analysis on each partition, where partitions with novel or unknown or ambiguous curves can be further analyzed to identify the amplified sequence. The selection of the partitions can be based on uncertainty scores, probability values assigned to the partitions based on melt curve analysis. In some case, the partitions can be selected based on outlier detection techniques.

Methods of sample pickup after digital assays proposed up to now are limited in the total number of partitions available, or in the density of the partitions. For instance, use of conventional microtiter plates such as 384-well plates allows easy pickup of samples by pipetting, but only hundreds or a few thousands of partitions are possible.

In some embodiments, after a sample is partitioned, each partition is subjected to a biological assay, such as the polymerase chain reaction (PCR). In many cases, it is desirable to perform further analysis on certain partitions based on the results of the first assay. For instance, it may be useful to perform Next-Generation Sequencing (NGS), or other sequencing methods such as Sanger Sequencing or shotgun sequencing, on the nucleic acid amplified and characterized in the first assay step in selected partitions in order to obtain the complete sequence of the amplicon. Other examples of biological assays that can be performed downstream after obtaining amplicons from a digital PCR reaction are library preparation for sequencing, Restriction Fragment Length Polymorphism (RFLP) Analysis to detect genetic mutations such as Single Nucleotide Polymorphism (SLP), mutation detection, molecular cloning, DNA methylation analysis, genotyping, in situ hybridization etc. To enable this further analysis, it is necessary to devise methods to selectively retrieve a product or substance from a specific partition while leaving the neighboring partitions untouched.

An additional challenge in retrieval is the removal of the desired product or substance from a partition without removing other undesired substances. For example, after creating the partitions, it is useful to isolate them from each other, and protect them from evaporation and the outside environment by using one or more protective layers. These layers could be composed of an immiscible liquid, such as an oil, mineral oil, silicone oil, fluorinated liquids such as FC-40 or HFE-7500, or water-immiscible esters. Other possible protective layers that could be applied are solid, such as polymers, including thermoplastic polymer films, polydimethylsiloxane, other types of rubber films or septa, metal coated polymer films, and so forth. It is desirable to retrieve a product generated in a partition without removing or contaminating it with the protective layer substance.

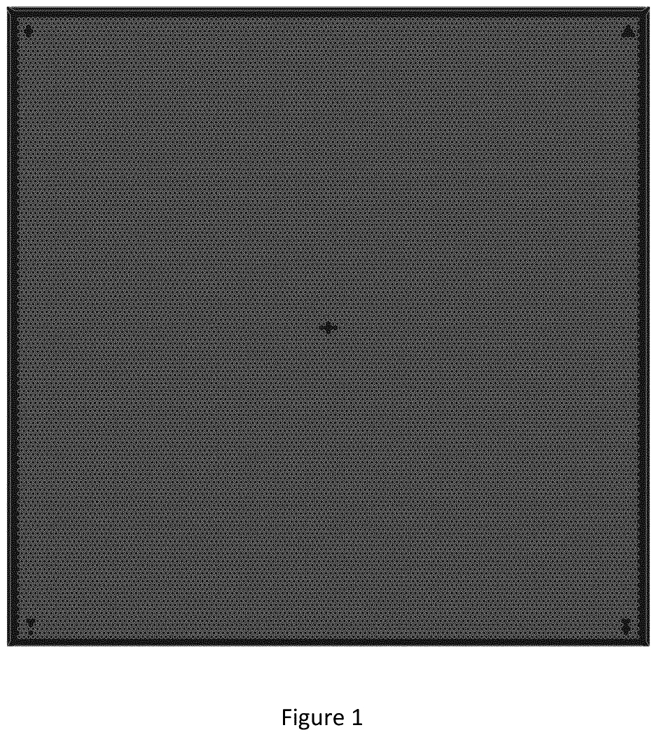

In addition, embodiments of the systems and methods provided herein describes retrieving samples from various fixed partition formats. In some embodiments, the fixed partition format is an array to partition aqueous samples using a silicon chip in which a dense array of high aspect ratio holes have been etched. In some embodiments, the holes in the single crystal silicon substrate were defined by photolithography followed by high-aspect plasma etching (also known as deep reactive ion etching (DRIE)). In some embodiments, the wall between the holes is thin, having a depth of about 5, 10, 15, 20, 25, 30, 35, 40, 45, or 50 μm, or a depth within a range defined by any two of the aforementioned values. In some embodiments, the thickness of the silicon substrate is about 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 μm, or a thickness within a range defined by any two of the aforementioned values. In some embodiments, the shape of the holes is hexagonal, and can include different dimensions, including for example: 52 μm side to side, with 20 micrometer walls; 70 μm side to side, with 24 μm walls; or 86 μm side to side, with 28 μm walls. These dimensions are exemplary, and other dimensions may be used. In some embodiments, dimensions between 50% and 60% of the total area of the outer surface of the substrate is devoted to holes. In some embodiments, the density is higher than what can be achieved with non-photolithographic methods, such as a density of at least 10%, 15%, 20%, 25%, 30%, 35%, 40%, 45%, or 50%, or a density within a range defined by any two of the aforementioned values. The full layout of one such design with 52 μm wells and 20 μm walls is depicted in FIG. 1.

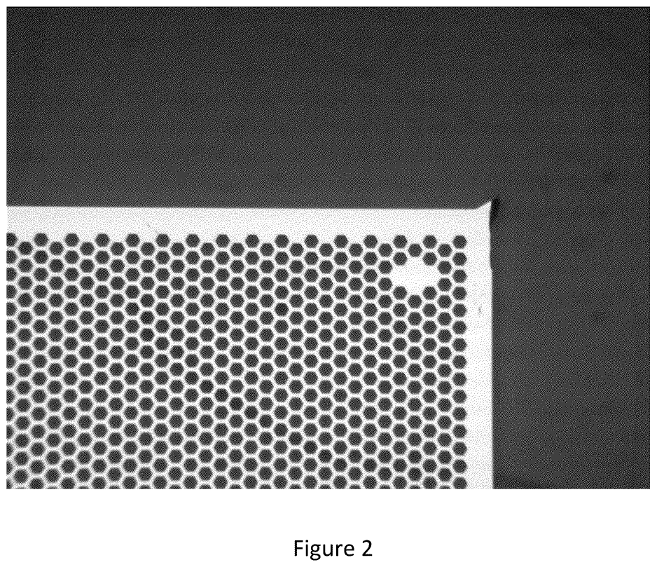

In some embodiments, the device contains at least 1,000 wells or partitions, such as at least 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 30,000, 35,000, 40,000, 50,000, 60,000, 70,000, 80,000, 90,000, 100,000, 200,000, 300,000, 400,000, 500,000, 600,000, 700,000, 800,000, 900,000, or 1,000,000 wells or partitions, or an amount within a range defined by any two of the aforementioned values. As an example, an array may include 21,383 wells in a 10 by 10 mm chip 300 μm thick, where each partition has a nominal volume of 702.5 picoliter. In the middle and in the corners of the silicon chip, groups of wells in a distinctive pattern have been suppressed to identify the chip type and its orientation. These patterns can also be used to help determine the location of wells in computer vision algorithms by providing a local reference in images at high enlargements. FIG. 2 depicts a microscope image at high enlargement of a portion of an etched silicon chip that includes such a reference pattern. As used herein, “computer vision system” refers to equipment, integrated circuit devices, or software that performs object recognition from digital images or video. In some implementations, a computer vision system can include software or hardware blocks performing respective tasks which can be organized as a graph. In some embodiments, the computer vision system uses a visual reference on the array. As used herein, “visual reference” refers to any indicator, marker, structure, or component designed, configured, placed, engineered, or organized to serve as a reference point or fixed known predetermined location for use in object recognition. A visual reference may be two-dimensional or three-dimensional. In certain embodiments, a visual reference may be a structure with patterned surfaces of known geometric dimensions with at least one surface visible to an image capture device/system. A visual reference may have a variety of shapes including cube, planar square, rectangle, triangle, tetrahedral, pyramid, or the like. The visual reference may have at least one surface visible by the computer vision system. Surfaces of a visual reference may include high contrast patterns. Moreover, a visual reference may have particular reflective coatings, radio opaque materials, and the like.

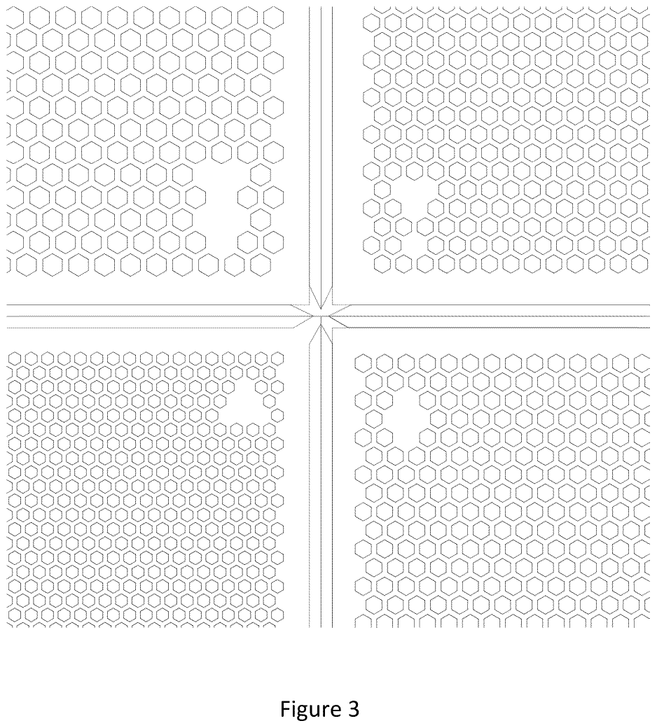

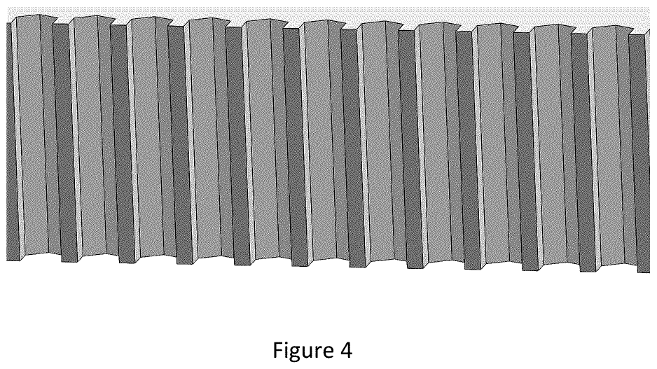

FIG. 3 depicts an embodiment of layout of an area of the photomask design where three design dimensions are present. As shown in FIG. 3, the design in the upper left corner has 86 μm hexagons, the two on the right have 70 μm hexagons, and the design on the lower left has 52 μm hexagons. Each design has a reference pattern in the corner. In each of these designs, the holes are filled by capillary action. This is possible because the holes are relatively narrow and have a high aspect ratio. FIG. 4 shows a cross-section of a group of holes 52 μm wide and separated by 20 μm walls.

In some embodiments, the array is generated using a fabrication process. In some embodiments, a final step in the fabrication process following a DRIE etch is stripping of all protective layers, including the silicon dioxide layer that was patterned to define the etch. The inside walls of these holes can include, for example, native oxide, since a thin layer of oxide is always present on the silicon surface. In other fabrication methods, the inner surfaces of the holes could be a thicker thermal oxide, with similar surface properties. In some embodiments, the filling of the holes is driven by capillary forces, also known as surface tension forces, which cause the small partition to fill when an aqueous solution is present at one of its openings. In some embodiments, the capillary forces are limited to the hole itself, since at the opening at the other end the capillary forces stop further expansion of the aqueous solution. Conversely, in order to remove a given partition from the hole in which it is located, a force must be applied to overcome the capillary retention force.

In general, the capillary pressure that causes the holes in the array to fill spontaneously with an aqueous solution are inversely proportional to the capillary dimension. This means that there is a trade-off between the ease of filling and the ease of partition retrieval. Smaller holes will have a higher capillary pressure that drives their filling, but that higher pressure may make it harder to retrieve their contents later. Larger holes will be more difficult to fill, but the vacuum needed to retrieve them will be lower.

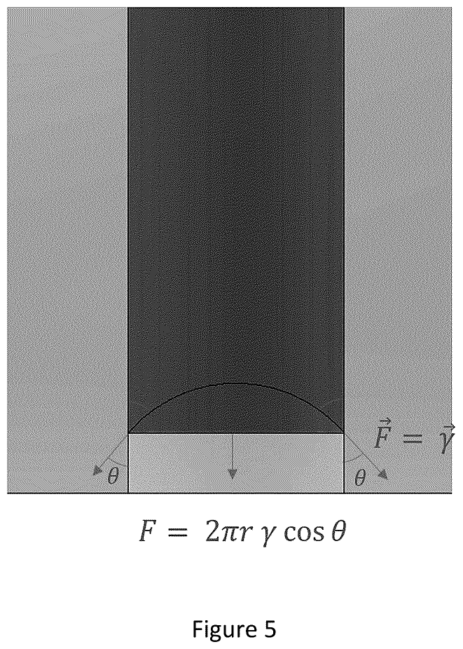

The capillary forces and pressures in the case of a cylindrical partition of height h and radius r can be calculated as follows. FIG. 5 shows an aqueous sample in the process of being removed in the upwards direction. The aqueous solution is replaced by another fluid, which may be air, or a protective fluid such as an immiscible liquid as described above.

Since the surface is hydrophilic, the contact angle θ is below 90 degrees, and the surface tension force is directed downwards. The surface tension force per unit length at the interface between the aqueous sample, the second immiscible fluid, and the wall of the partition is given by \vec{F}=\vec{y}, and is parallel to the interface, with an angle of θ relative to the axis of the cylinder. By integrating over the perimeter of the circular interface, the total force along the axis is given by:

$$F = 2 π r γ cos θ \qquad ( 1 )$$

This force divided by the area πr² of the partition is the capillary pressure given by:

$${P}_{cap} = \frac{2 γ cos θ}{r} \qquad ( 2 )$$

This is the familiar Young-Laplace equation, and it gives the externally applied pressure difference needed to overcome the capillary forces that retain a partition, and allow its retrieval. To move the partition out in the geometry of FIG. 5, the force must be upwards, which means the pressure must be positive below the sample, or a vacuum above the sample. Note that the Laplace pressure in equation (2) is the pressure or vacuum needed to balance the capillary forces. A larger pressure or vacuum will be needed to move the sample and overcome the viscous forces involved. Therefore equation (2) defines the minimal retrieval pressure in the case of a cylinder with a circular cross-section.

These equations can be generalized to geometries other than a cylinder with a circular cross-section. In the case of a cylinder with arbitrary cross-section, equation (1) is replaced by a line integral along the perimeter, and the total force becomes:

$$F = {\int }_{s} γ cos θ ds \qquad ( 3 )$$

and the capillary pressure is obtained by dividing that force by the area A of that cross-section:

$${P}_{cap} = \frac{{\int }_{s} γ cos θ ds}{A} \qquad ( 4 )$$

This is the minimal retrieval pressure for a cylinder with arbitrary cross-section. In particular, for a hexagon with side a, the perimeter is 6a, the capillary force is

$$F = 6 a γ cos θ \qquad ( 5 )$$

and by dividing by the area of the hexagon obtains the minimal retrieval pressure for a cylinder with hexagonal cross-section:

$${P}_{cap} = \frac{4}{\sqrt{3}} \frac{γ cos θ}{a} \qquad ( 6 )$$

For example, in the case of a hexagon with side-to-side dimensions of 52 μm, the side is a=52√(3)=30 μm, and this is the value to be used in equation (6).

To completely remove the sample from the well with height h a force at least equal to F must be applied over a distance h. The work performed in the process is Fh, and represents the capillary retention energy of the sample in the well. The equation of this retention energy follows from equation (1), namely:

$${E}_{ret} = 2 π r γ cos θ h \qquad ( 7 )$$

The capillary retention energy from equation (7) can also be written in the form:

$${E}_{ret} = ( \frac{2 γ cos θ}{r} ) ( π {r}^{2} h ) = {P}_{cap} {V}_{part} \qquad ( 8 )$$

where Vₚₐᵣₜ is the volume of the partition.

When formulated as a P Vₚₐᵣₜ product, the expression of the retention energy can be generalized to include contributions from viscous forces, namely the pressure needed to flow the partition volume through the partition itself, and the subsequent geometry of the pickup device. This pressure is given by the Hagen-Poiseuille law for cylindrical configurations, and can be calculated by various means for other geometries. The more general retention energy is then given by:

$${E}_{ret} = {P}_{cap} {V}_{part} + {P}_{visc} {V}_{part} \qquad ( 9 )$$

where P_{visc} is the pressure from viscosity needed to move the partition volume in a given time. In some embodiments, the component involves the time available or desirable for sample retrieval.

For example, the capillary pressure can be calculated for the array of holes in silicon in the geometry described above in FIG. 4. The hexagonal openings have a side of 30 μm. Assuming that an oil phase is used as a protective fluid, and that the aqueous phase is replaced by this fluid, such as HFE-7500 with an added detergent, which is commonly used for partitioning in digital PCR. According to the literature, the surface tension of the HFE 7500/water interface is 26 mN/m. The contact angle of the oil/water interface at a silicon surface is not known from the literature but must be hydrophilic to enable capillary filling of the partitions. The angle must therefore be between 0 and 90 degrees, such as 45 degrees. When substituting these numbers into equation (6) a value of P_{cap}=2001 Pa. This is the minimum retrieval pressure for this geometry and these fluids. When other fluorinated liquids such as FC-40 are used instead of HFE-7500 this value may be similar in magnitude, since fluorinated oils have similar properties.

Further, to remove a particular sample from the partition where it is located, an energy at least equal to the retention energy must be focused on that sample, using a mechanism such as a needle probe, focused optical energy, or focused acoustic energy. This mechanism must also be capable of collecting the sample while penetrating any protective layer that surrounds it, such as an immiscible fluid, without losing the integrity of the partition. One way of applying the needed energy to retrieve a partition is to apply a focused vacuum exerting a pressure greater than the retention pressure on one side of the array with a pickup probe, for a time sufficient to retrieve the entire sample. Conversely, the application of a focused pressure on the other side of the partition will cause its expulsion into the protective oil layer, where it can then be retrieved.

In some embodiments, a vacuum that must be applied to a fine needle probe must be higher than the minimum retrieval pressure given by equation (4) in order to cause the partition to flow out of the hole in the silicon and into the needle probe. The vacuum necessary for this can be estimated in the same conditions as were used to estimate the capillary pressure. In some embodiments, given a probe length of 1 cm, and a diameter 40 μm, as it will need to be smaller than the size of the partition (a hexagon 52 μm side by side). The flow resistance of this needle for water can then be calculated using the Hagen-Poiseuille law. In some embodiments, the partition volume is in the order of a nanoliter (depending on the thickness of the silicon), and the desired time for retrieval is one second. That leads to a flow rate of 10⁻⁶ cm³/s, and when multiplied by the flow resistance the result is a required additional vacuum of 2266 Pa. This is about equal to the capillary pressure, and therefore a good estimate is that retrieval of a partition by a needle probe will require a vacuum of about twice the minimum retrieval pressure given by equation (4), and at least 50% higher if slower retrieval is acceptable.

In some embodiments, the retrieval process can be completely automated such as by using a computer controlled 2-dimensional or 3-dimensional micro manipulator retrofitted to the vacuum-capillary assembly for sample pick up. An image analysis algorithm, such as the one proposed in Sinha et al., SLAS Technologies, 2018, can map the location (x-y coordinates) of all the partitions relative to a reference marker in a particular image. Then, using any of the methods described herein to identify the partition or partitions to be retrieved, a list of coordinates can be transferred to the micro-manipulator controller software which can automatically position the retrieval assembly on top of the partition and then recover the sample using a combination of automated z movement and capillary pressure.

Some embodiments provided herein relate to the following enumerated alternatives:

1. A method for biological analysis of a sample, the method comprising: providing a substrate comprising an array comprising individual partitions, each partition comprising a hole suitable to allow filling of each partition by capillary force; dividing the sample by loading an aliquot of the sample into the individual partitions; performing a biological analysis on each partition; identifying one or more individual partitions of interest based on the biological analysis; and retrieving the contents of the one or more partitions of interest.

2. The method of claim 1, wherein the array comprises at least 10,000 individual partitions.

3. The method of any one of alternatives 1-2, wherein retrieving the contents of the one or more partitions of interest comprises application of a focused vacuum or pressure device.

4. The method of alternative 3, wherein the focused vacuum or pressure device exerts a retrieval pressure on the partition greater than a capillary retention pressure of the partition, and wherein the retrieval pressure is calculated based on a dimension of the partition.

5. The method of alternative 4, wherein the retrieval pressure is equal to or greater than a minimal retrieval pressure.

6. The method of alternative 5, wherein the retrieval pressure is at least 50% higher than the minimal retrieval pressure.

7. The method of alternative 5, wherein the retrieval pressure is at least 100% higher than the minimal retrieval pressure.

8. The method of alternative 3, wherein the vacuum or pressure device comprises a probe having dimensions smaller than the dimensions of the partition.

9. The method of alternative 4, wherein the focused vacuum or pressure device comprises a probe or a needle for applying the retrieval pressure.

10. The method of any one of alternatives 1-9, wherein the individual partitions are circular.

11. The method of any one of alternatives 1-9, wherein the individual partitions are hexagonal.

12. The method of any one of alternatives 1-11, further comprising applying a protective layer after step dividing the sample.

13. The method of alternative 12, wherein the protective layer comprises a water-immiscible liquid.

14. The method of alternative 12, wherein the protective layer comprises a fluorinated oil.

15. The method of any one of alternatives 12-14, wherein the protective layer further comprises one or more polymer layers.

16. The method of any one of alternatives 1-15, wherein the individual partitions have an area density of at least 30%.

17. The method of any one of alternatives 1-16, wherein the biological analysis comprises nucleic acid amplification.

18. The method of any one of alternatives 1-17, wherein the biological analysis comprises melt curve analysis.

19. The method of any one of alternatives 1-18, wherein the sample comprises whole cells or microorganisms.

20. The method of any one of alternatives 1-19, wherein a location of the individual partition of interest is determined by a computer vision system.

21. The method of alternative 20, wherein the computer vision system utilizes one or more visual references in the array.

22. A method of recovering selected partitions in a digital assay, the method comprising: providing a substrate comprising an array comprising individual partitions, each partition comprising a hole suitable to allow filling of each partition by capillary force; dividing the sample by loading an aliquot of the sample into the individual partitions; performing a first biological analysis on each partition; identifying one or more individual partitions of interest based on the first biological analysis; and performing a second biological analysis on the one or more individual partitions of interest based on the first biological analysis.

23. The method of alternative 22, wherein the first biological analysis comprises a digital PCR assay followed by melt curve analysis.

24. The method of any one of alternatives 22-23, wherein the second biological analysis comprises conventional qPCR.

25. The method of any one of alternatives 22-24, wherein the array comprises at least 10,000 individual partitions.

26. The method of any one of alternatives 22-25, further comprising retrieving the contents of the one or more partitions of interest.

27. The method of alternative 26, wherein retrieving the contents comprises selectively retrieving DNA from a sample from a single partition of interest.

28. The method of any one of alternatives 26-27, wherein retrieving the contents of the one or more partitions of interest comprises application of a focused vacuum or pressure device.

29. The method of alternative 28, wherein the focused vacuum or pressure device exerts a retrieval pressure on the partition greater than a capillary retention pressure of the partition, and wherein the retrieval pressure is calculated based on a dimension of the partition.

30. The method of alternative 29, wherein the retrieval pressure is equal to or greater than a minimal retrieval pressure.

31. The method of alternative 30, wherein the retrieval pressure is at least 50% higher than the minimal retrieval pressure.

32. The method of alternative 31, wherein the retrieval pressure is at least 100% higher than the minimal retrieval pressure.

33. The method of any one of alternatives 28-32, wherein the vacuum or pressure device comprises a probe having dimensions smaller than the dimensions of the partition.

34. The method of any one of alternatives 28-33, wherein the focused vacuum or pressure device comprises a probe or a needle for applying the retrieval pressure.

35. The method of any one of alternatives 22-34, wherein the individual partitions are circular.

36. The method of any one of alternatives 22-34, wherein the individual partitions are hexagonal.

37. The method of any one of alternatives 22-36, further comprising applying a protective layer after dividing the sample.

38. The method of alternative 37, wherein the protective layer comprises a water-immiscible liquid.

39. The method of alternative 37, wherein the protective layer comprises a fluorinated oil.

40. The method of any one of alternatives 37-39, wherein the protective layer further comprises one or more polymer layers.

41. The method of any one of alternatives 22-40, wherein the individual partitions have an area density of at least 30%.

42. The method of any one of alternatives 22-41, wherein the biological analysis comprises nucleic acid amplification.

43. The method of any one of alternatives 22-42, wherein the biological analysis comprises melt curve analysis.

44. The method of any one of alternatives 22-43, wherein the sample comprises whole cells or microorganisms.

45. The method of any one of alternatives 22-44, wherein a location of the individual partition of interest is determined by a computer vision system.

46. The method of alternative 45, wherein the computer vision system utilizes one or more visual references in the array.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1

The following example demonstrates a comparison between blunt and sharper tip for the retention of the content of the etched silicon chip wells during digital assay.

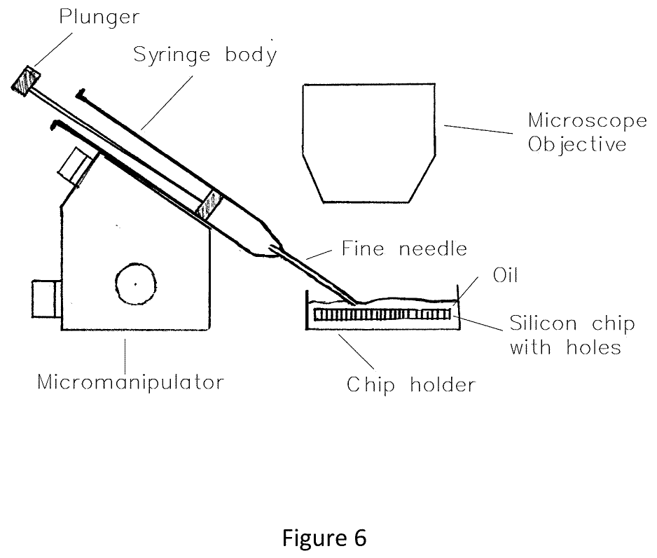

A QuantStudio silicon chip (Thermo-Fisher) was mounted in the device holder. The dimensions of the device are very close to those of the design shown in FIG. 1, namely hexagons 52 micrometers side to side, and a wall of 20 micrometers between them. The overall chip has dimensions of 10 by 10 mm, and contains a total of about 20,400 wells. The wells were manually filled with a filling tool with 15 microliters of a ROX fluorescent dye stock solution, diluted 5:1 in a PBS solution. After the filling, several hundred microliter of QuantStudio partitioning fluid (a fluorinated oil) were pipetted over the chip, in order to cover the top surface completely with a layer of oil. Samples were removed with fine needle probes of various types connected by tubing to a syringe with a plunger, as shown in FIG. 6. A micromanipulator was used for fine control of the positioning of the needle probe, together with a stereoscopic microscope to observe the exact location of the needle tip relative to each partition. Evaluation of the results of the experiments was done with an Olympus MVX microscope with fluorescence imaging at the wavelengths for ROX dyes. This way the presence or absence of the aqueous phase can be sensitively determined.

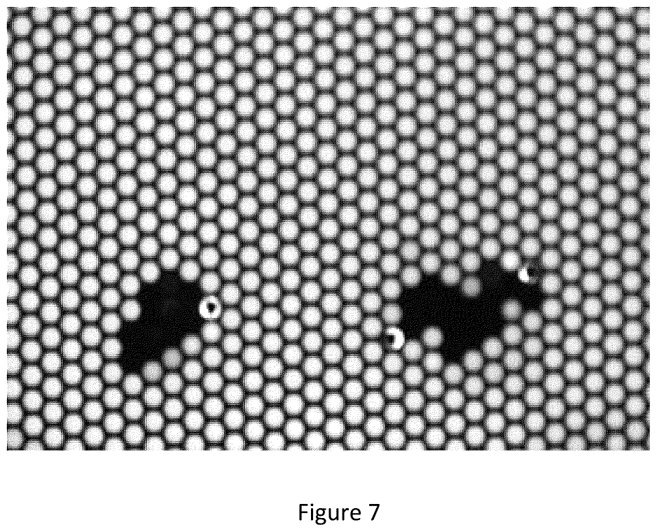

A glass pickup probe with a relatively blunt tip that was larger than the size of the partitions was used and therefore was unable to penetrate the partition directly. After positioning the probe near a particular partition, vacuum is applied by pulling the syringe plunger out for a certain distance. This creates a vacuum that is not measured or controlled. The action of pulling out the syringe also caused movement of the glass probe tip that made controlling its precise location difficult. The result was that it was possible to remove clusters of wells, but not single wells, as shown in FIG. 7.

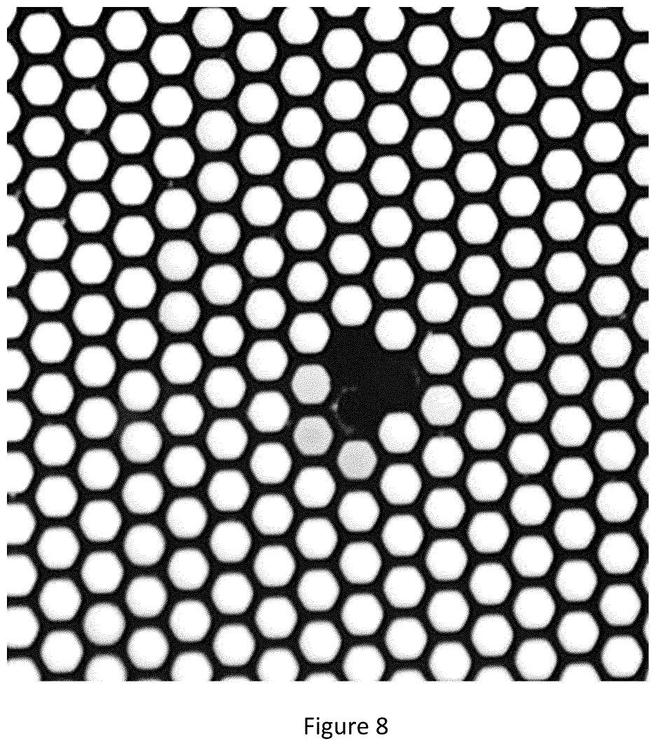

In two separate instances, a group of 10 to 20 wells were picked up simultaneously, but it was not possible to retrieve a single partition. Then, a sharper tip was used with tip smaller than the size of the partitions was used and was able to move closer to a given partition, and in some instance even inside it. The vacuum was again applied by the use of a syringe plunger. The result is shown in FIG. 8. Hence, the finer tip was able to retrieve a smaller cluster of wells, namely three wells, but not a single well.

### Example 2

The following example demonstrates a comparison between an uncontrolled vacuum source and miniature vacuum pump on the retention of the content of the etched silicon chip wells during digital assay.

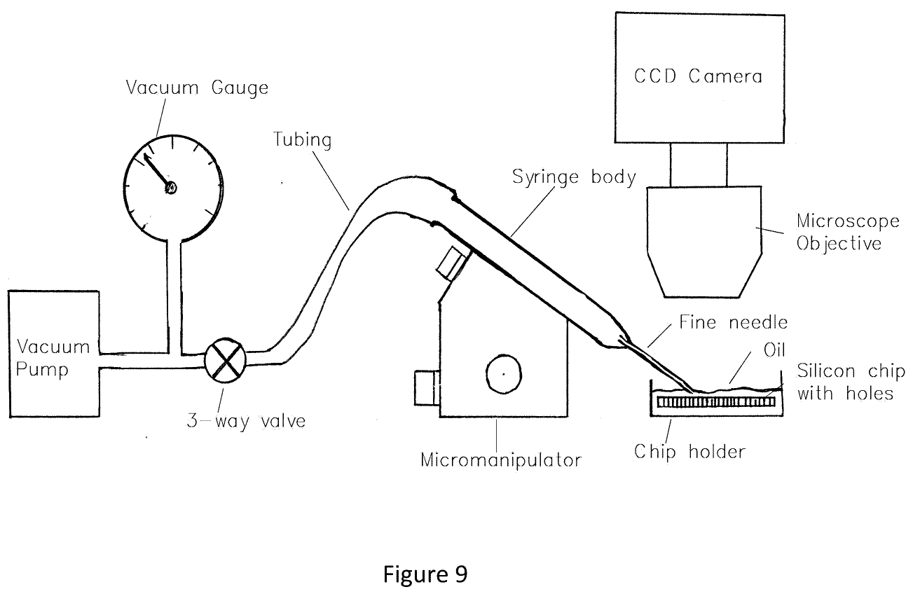

An uncontrolled vacuum source was replaced by a miniature vacuum pump connected to a vacuum gauge and a three-way valve. This allowed us to measure the vacuum being used, and the valve allowed adjusting the length of time during which it was applied. The value of the vacuum could be controlled by various means, such as adjusting the voltage applied to the vacuum pump, or by the use of a vacuum regulator (see FIG. 9). In the experiments described below, the vacuum was controlled to a value of about 60 kPa by adjusting the voltage applied to the vacuum pump. This value is well above the typical values of the estimated capillary pressure.

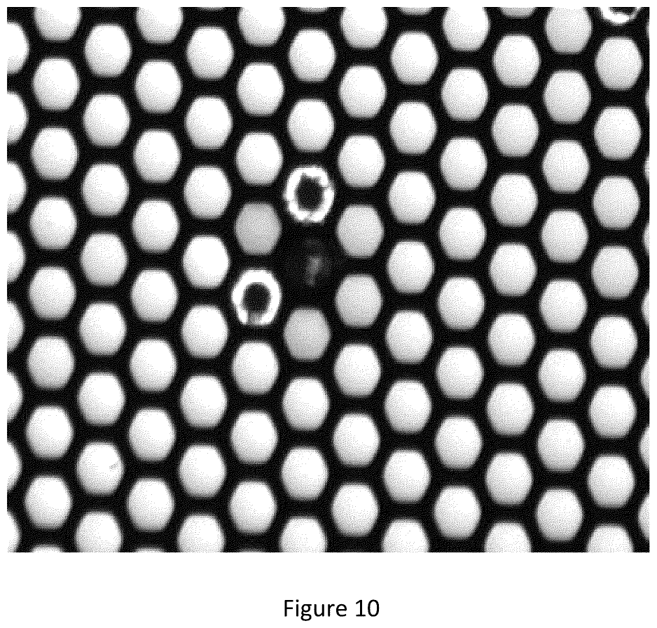

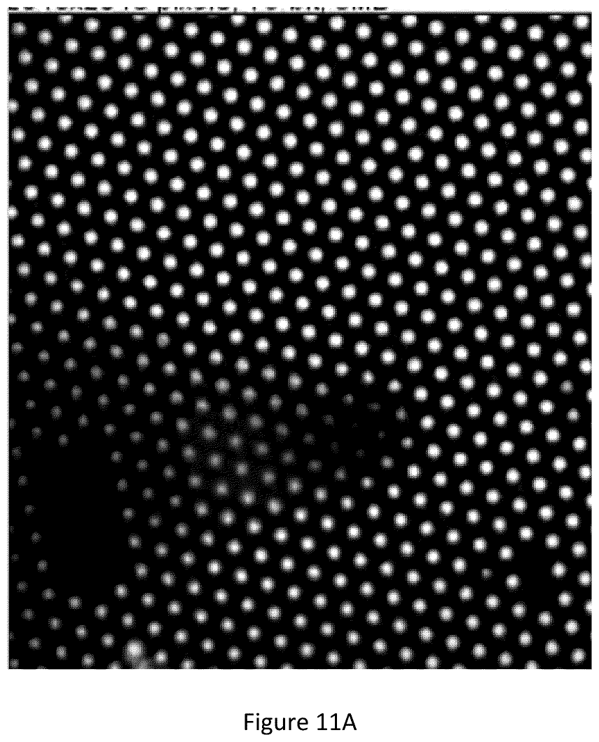

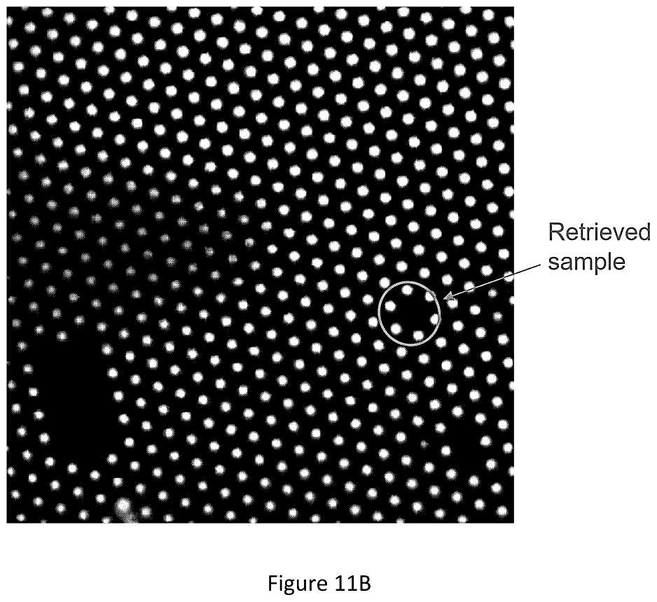

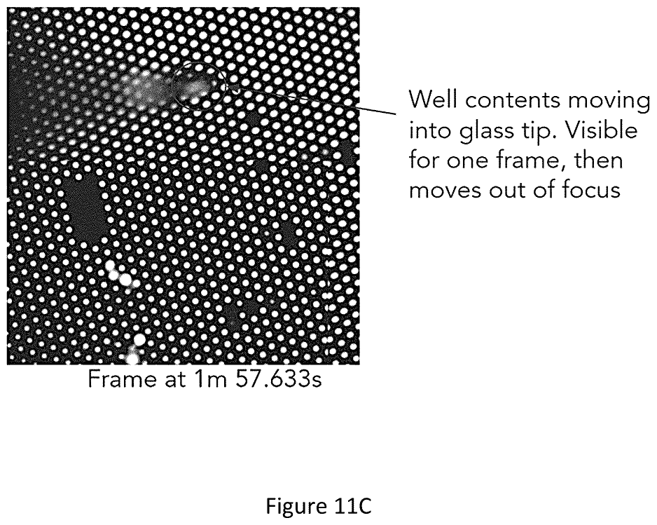

When sample pickup was performed with this experimental configuration, a single partition was completely picked up, together with a partial pickup of two neighboring wells, FIG. 10. In further experiments, the positioning of the probe was adjusted, together with the timing of the vacuum, to allow improved selective pickup of a single partition without affecting the neighboring partitions. FIG. 11A shows an array filled with fluorescent ROX dye, before a partition is retrieved. The outline of the needle is out of focus, but darkens a group of wells in the lower middle.

After the pickup and the retraction of the needle, it can be seen that a single well is picked up without affecting the neighboring wells, FIG. 11B. The needle outline is still faintly visible to the left and slightly above the picked well, FIG. 11C.

Experiments were also performed with no vacuum applied, in which case only capillary forces from the pickup probe or needle could drive the retrieval of a partition. This was attempted both with glass needles, and with very fine stainless-steel needles (34 gauge). In all cases, no partition retrieval was observed. This shows that a minimum vacuum is required for sample pickup, as is predicted by equation (6).

### Example 3

The following example demonstrated the effect of not using oil covering layer on the recovery of partitions.

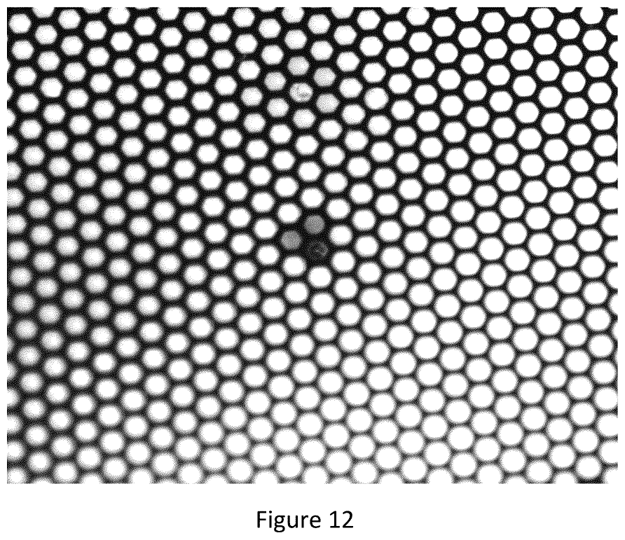

It was observed that sample retrieval was still possible, but time is limited before some of the sample is lost to evaporation. The sample pickup was not as selective as in the case that an oil protective layer was present; one partition was mostly picked up, but a portion of two neighboring ones were picked up as well, FIG. 12. In the partition that was mostly picked up, a small amount of sample can be seen to remain on the edges of the well. This part of the sample could be held in place by the hydrophilicity of the well surface, relative to air. This indicates that it could be difficult to pick up a sample completely in the absence of an immiscible fluid other than air to replace it. In the previous examples where the samples are surrounded by oil, it appears to be easier to replace the sample that is picked up by oil than it is to replace it with air. This is another advantage of the use of a protective layer such as an immiscible liquid.

Some experiments included a reference pattern in the well pattern that can be used to track the well location that is being picked up. This is useful, because there typically are many thousands of wells in a dense array, and to position the pickup probe accurately, a high magnification is needed. In high magnification images it can be difficult to identify the precise location of a well in the absence of a reference pattern in the image. With a reference pattern of some type, it can still be possible to determine the exact coordinates of the well that is picked up.

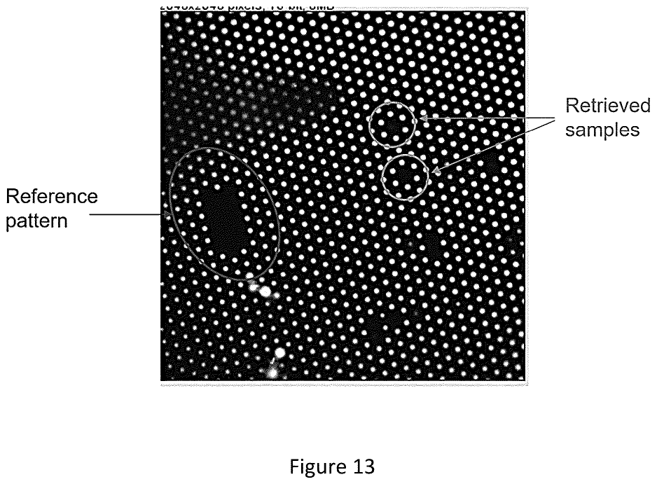

FIG. 13 shows a reference pattern on the silicon chip consisting of a group of locations in the array where the wells have not been etched, and where there is no fluorescence visible as a result. This group has the shape of a chevron. The locations of two of the retrieved samples are also shown. It can be seen that in this frame the precise coordinates of these locations relative to any part of the reference pattern can be determined simply by counting. Since the absolute location of the reference pattern in the array is also known, the absolute location of the samples that have been picked up can then also be determined.

### Example 4

The following example demonstrates performing a first assay on all the partitions of a fixed-partition digital PCR chip, and using the results of the first assay to identify and locate certain partitions of interest, retrieving some of those identified partitions, and performing a second assay on those retrieved partitions.

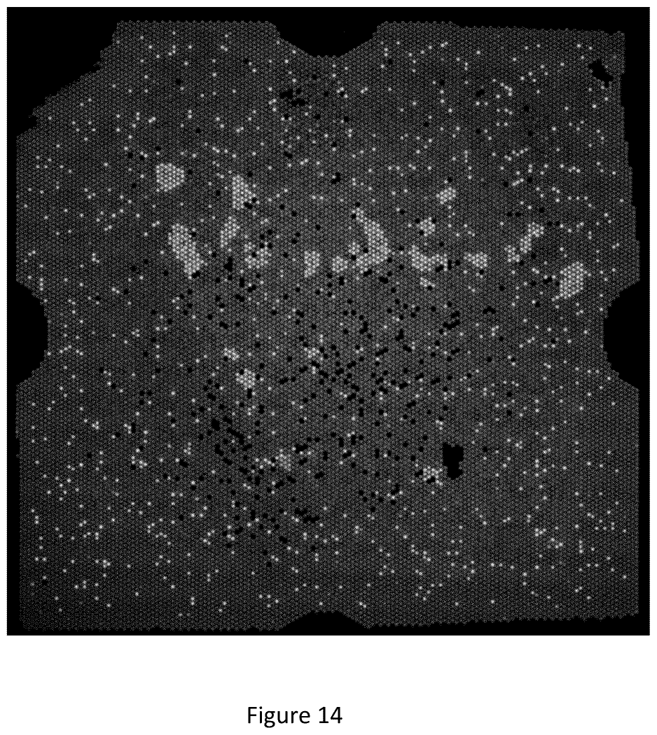

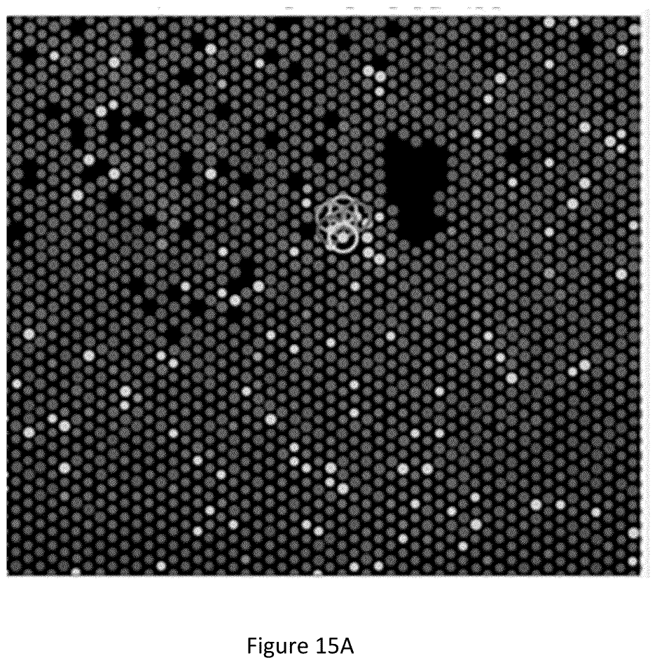

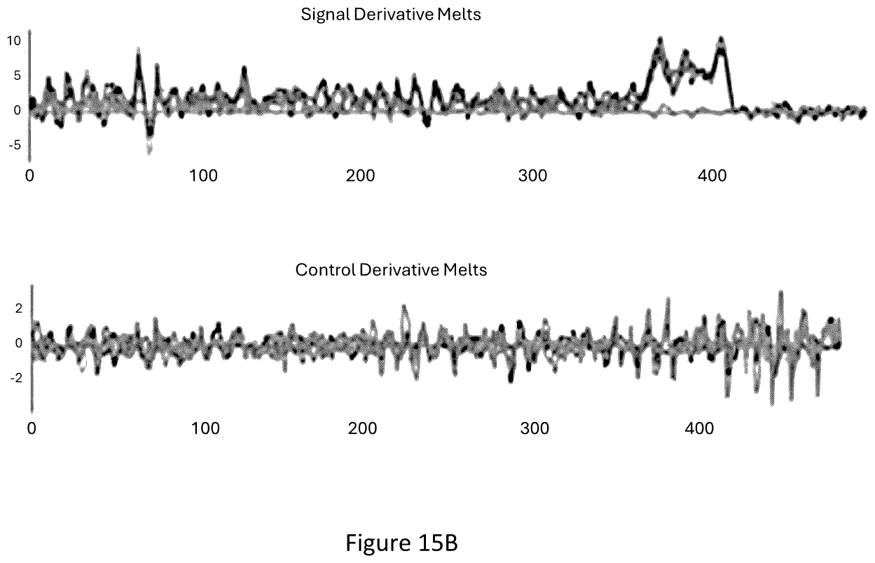

A digital PCR assay was performed followed by a melt curve analysis with a QuantStudio silicon chip (ThermoFisher) of the type described in Example 1. A sample containing intact Candida parapsilosis fungi, together with PCR master mix containing an intercalating dye, and universal primers targeting bacteria and fungi was partitioned into the QuantStudio chip, covered with the partitioning oil, and thermally cycled to amplify the target sequence. After thermal cycling, a melt curve analysis was performed to determine which partitions contained fungi. FIG. 14 shows the fluorescence of the EvaGreen intercalating dye. The wells where amplification occurred are strongly fluorescent. A cluster of positive wells close to the location marker present in the QuantStudio chip was identified for purposes of subsequent sample retrieval. These wells are circled in FIG. 15A, and the associated melt curves in these wells are shown in FIG. 15B. The curves labeled “Signal Derivative Melts” show a distinctive pattern that is characteristic for C. parapsilosis.

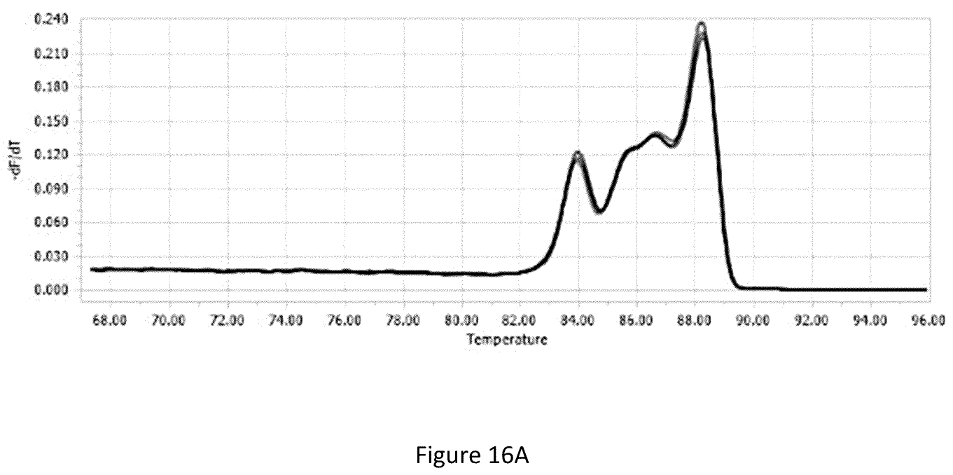

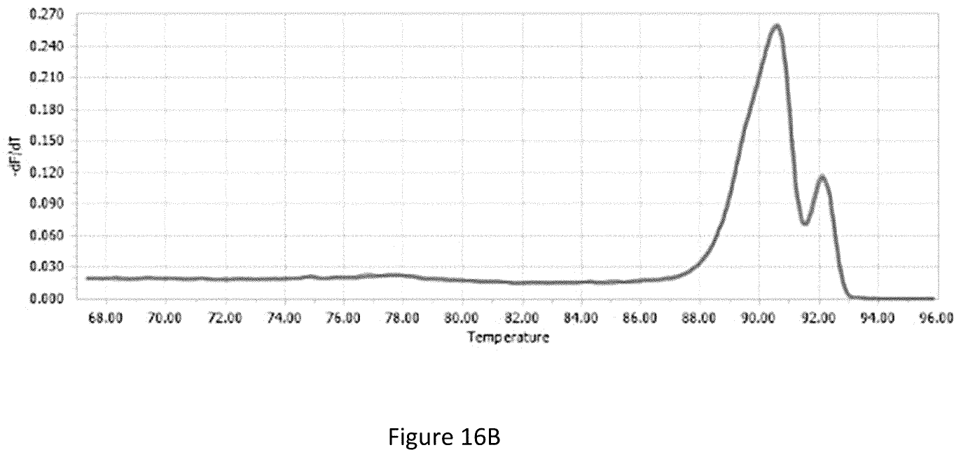

Eight of the wells in the cluster shown were retrieved from the silicon chip with a setup similar to that shown in FIG. 9, with a syringe pump instead of the vacuum pump and vacuum gauge. This was done to allow controlled fluid flow in both directions. For each well retrieval, the following procedure was followed: (a) using a micromanipulator to insert a glass needle with a sharp tip into a selected partition; (b) using the syringe pump to aspirate the fluid from the partition into the glass needle; (c) moving the glass needle to a reservoir of deionized water; (d) using the syringe pump to aspirate approximately 10 μl of water into the glass needle, where it is combined with the material previously aspirated; (e) moving the glass needle into a PCR tube; (f) using the syringe pump to dispense the entire contents of the glass needle into the PCR tube. A conventional qPCR was performed in each retrieved sample by adding the necessary reagents and primers, and thermally cycling in a LightCycler 96° C. (Roche) instrument. Additionally, controls with cyanobacteria DNA were thermally cycled at the same time. After amplification, melt curve analysis was performed to identify the type of DNA that was amplified. In FIG. 16A are shown the melt curves for the eight samples retrieved from the positive wells in the cluster shown in FIG. 15A. They show the same characteristic shape for C. parapsilosis as we found earlier in the first assay (FIG. 15B). In FIG. 16B are shown the melt curves for the control with cyanobacteria DNA. These curves have a different characteristic shape, and a peak at higher temperature, which is expected for cyanobacteria DNA. This shows that the ability to selectively retrieve the DNA from a single partition, with a volume of less than a nanoliter, and performing a DNA analysis on it is able to identify the type of DNA originally present in the sub-nanoliter partition.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments.

What is claimed is:

1. A method for biological analysis of a sample, the method comprising:
(a) providing a substrate comprising an array comprising individual partitions, each partition comprising a hole suitable to allow filling of each partition by capillary force;
(b) dividing the sample by loading an aliquot of the sample into the individual partitions;
(c) performing a biological analysis on each partition;
(d) identifying one or more individual partitions of interest based on the biological analysis; and
(e) retrieving the contents of the one or more partitions of interest.

2. The method of claim 1, wherein the array comprises at least 10,000 individual partitions.

3. The method of claim 1, wherein retrieving the contents of the one or more partitions of interest comprises application of a focused vacuum or pressure device.

4. The method of claim 3, wherein the focused vacuum or pressure device exerts a retrieval pressure on the partition greater than a capillary retention pressure of the partition, and wherein the retrieval pressure is calculated based on a dimension of the partition.

5. The method of claim 3, wherein the vacuum or pressure device comprises a probe having dimensions smaller than the dimensions of the partition.

6. The method of claim 4, wherein the focused vacuum or pressure device comprises a probe or a needle for applying the retrieval pressure.

7. The method of claim 1, wherein the individual partitions are circular or hexagonal.

8. The method of claim 1, further comprising applying a protective layer after step (b).

9. The method of claim 8, wherein the protective layer comprises a water-immiscible liquid, a fluorinated oil, or one or more polymer layers.

10. The method of claim 1, wherein the biological analysis comprises nucleic acid amplification or melt curve analysis.

11. The method of claim 1, wherein the sample comprises whole cells or microorganisms.

12. The method of claim 11, wherein the computer vision system utilizes one or more visual references in the array.

13. A method of recovering selected partitions in a digital assay, the method comprising:
providing a substrate comprising an array comprising individual partitions, each partition comprising a hole suitable to allow filling of each partition by capillary force;
dividing the sample by loading an aliquot of the sample into the individual partitions;
performing a first biological analysis on each partition;
identifying one or more individual partitions of interest based on the first biological analysis; and
performing a second biological analysis on the one or more individual partitions of interest based on the first biological analysis.

14. The method of claim 13, wherein the first biological analysis comprises a digital PCR assay followed by melt curve analysis.

15. The method of claim 13, wherein the second biological analysis comprises conventional qPCR.

16. The method of claim 13, further comprising retrieving the contents of the one or more partitions of interest.

17. The method of claim 16, wherein retrieving the contents comprises selectively retrieving DNA from a sample from a single partition of interest.

18. The method of claim 16, wherein retrieving the contents of the one or more partitions of interest comprises application of a focused vacuum or pressure device.

19. The method of claim 18, wherein the focused vacuum or pressure device exerts a retrieval pressure on the partition greater than a capillary retention pressure of the partition, and wherein the retrieval pressure is calculated based on a dimension of the partition.

20. The method of claim 18, wherein the vacuum or pressure device comprises a probe having dimensions smaller than the dimensions of the partition.
